(19) United States
(12) Patent Application Publication
(10) Pub. No.: US 2010/0179390 A1
(43) Pub. Date: Jul. 15, 2010

(54) COLLABORATIVE TABLETOP FOR CENTRALIZED MONITORING SYSTEM

(75) Inventor: Carl Claude Davis, Menomonee Falls, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/352,376

(22) Filed: Jan. 12, 2009

(51) Int. Cl. A61B 5/00; G06F 17/30; G06G 7/60

(52) U.S. Cl. 600/301; 702/19

(57) ABSTRACT

In one embodiment, a centralized monitoring system for monitoring at least one patient is described. The centralized monitoring system comprises a processor configured for receiving patient data from a plurality of patient monitoring systems, a collaborative tabletop coupled to the processor, the collaborative tabletop configured to visually convey the patient data, at least one high resolution display configured to display patient data associated with a plurality of patients and an input device coupled to the processor, the input device configured for inputting at least one of textual data, graphical data and image data associated with the patient. The collaborative tabletop comprises a main display surface, an information screen and a reference screen.

### FIELD OF INVENTION

The invention generally relates to patient monitoring and more particularly to a centralized patient monitoring system using a collaborative tabletop.

### BACKGROUND OF THE INVENTION

A centralized monitoring area in a healthcare establishment includes facilities for monitoring a large number of patients. In the centralized monitoring area a telemetry technician views the patients at a central location. Centralized monitoring is implemented by using a plurality of screens in a standard vertical orientation. Within the centralized monitoring area, each technician is provided with his own screen. This approach is used universally in patient monitoring. However, this approach limits the ability of multiple technicians to manage a single patient.

Another limitation associated with the current model relates to working with multiple screens. This requires a user to either have a single mouse move across a number of screens or leverage touch on a vertical screen. The former can lead to poor situational awareness and the latter can lead to fatigue.

Several solutions provided in the prior art describe table based displays and input for computer based screens. Each of the solutions treat the table as a large display with computer images having touch capability. However, these general solutions fail to meet specialized application needs where resolution and workflow are critical. For example these solutions comprise information about a single resolution for data and fail to display specialized data.

Hence there exists a need for a centralized monitoring system capable of manipulating resolution and workflow.

### BRIEF DESCRIPTION OF THE INVENTION

The above-mentioned shortcomings, disadvantages and problems are addressed herein which will be understood by reading and understanding the following specification.

In one embodiment, a centralized monitoring system for monitoring at least one patient is described. The centralized monitoring system comprises a processor configured for receiving patient data from a plurality of patient monitoring systems, a collaborative tabletop coupled to the processor, the collaborative tabletop configured to visually convey the patient data, at least one high resolution display configured to display patient data associated with a plurality of patients and an input device coupled to the processor, the input device configured for inputting at least one of textual data, graphical data and image data associated with the patient.

In another embodiment, a collaborative tabletop for a centralized monitoring system is provided. The collaborative tabletop comprises a main display surface for presenting patient data, the main display surface configured to be used by a plurality of users and at least one information screen configured to be used by a single user.

In yet another embodiment, a method of displaying patient data in a collaborative tabletop is provided. The method comprises implementing a main display surface for presenting patient data, the main display surface configured to be used by a plurality of users and implementing at least one information screen configured to be used by a single user.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of a hospital network, in one embodiment;

FIG. 2 shows a block diagram of a centralized monitoring system, in one embodiment;

FIG. 3 shows a block diagram of a collaborative tabletop employed in the centralized monitoring system shown at FIG. 2; and

FIG. 4 shows a schematic diagram of the centralized monitoring system shown at FIG. 2.

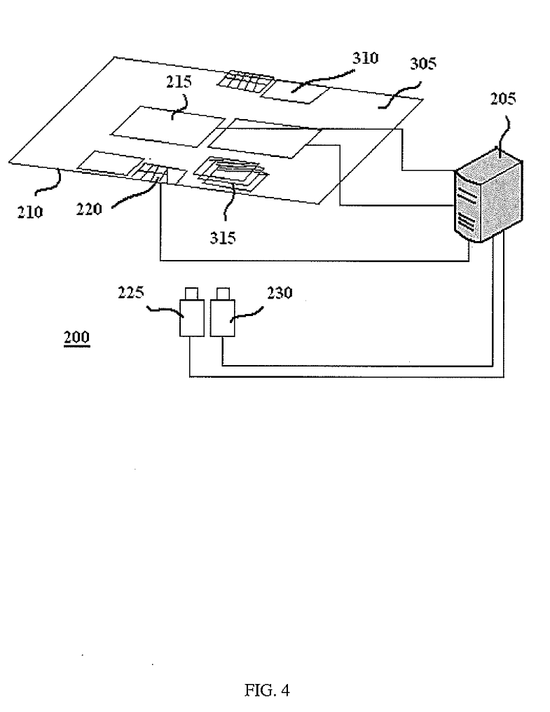

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

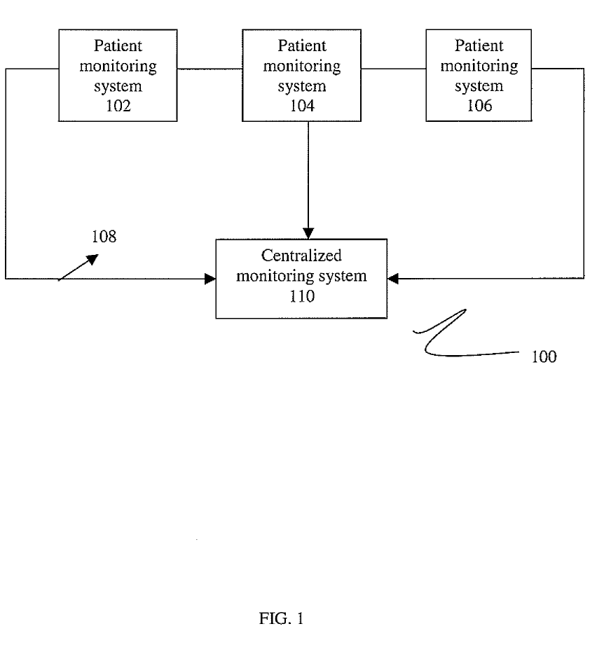

As depicted in FIG. 1, a hospital network 100 is shown comprising a plurality of patient monitoring systems 102, 104 and 106. The hospital network 100 can be interconnected via a communication link 108, which is a hard-wired connection (e.g., cable, bus, etc.) or a wireless connection (e.g., infrared, radio frequency, etc.) or a combination thereof. The patient monitoring systems 102, 104 and 106 are connected to each other and to a centralized patient monitoring system 110 via the communication link 108. The centralized patient monitoring system 110 (referred to hereafter as centralized monitoring system 110) enables a plurality of technicians to collectively monitor a large number of patients from a single location. In some applications, each technician can monitor approximately up to one hundred patients.

The patient monitoring systems 102, 104 and 106 are each configured to monitor one of the patients, to generate patient data based on specific characteristics being monitored, and to transmit the patient data to the centralized monitoring system 110. In a non-limiting manner, the patient monitoring systems 102, 104 and 106 may each comprise an electrocardiograph, a blood pressure monitor, a thermometer, and/or a pulse oximeter. Correspondingly, the patient data generated by the patient monitoring systems 102, 104 and 106 may comprise electrocardiogram (ECG) data, blood pressure data, temperature data, oxygen saturation and/or pulse data.

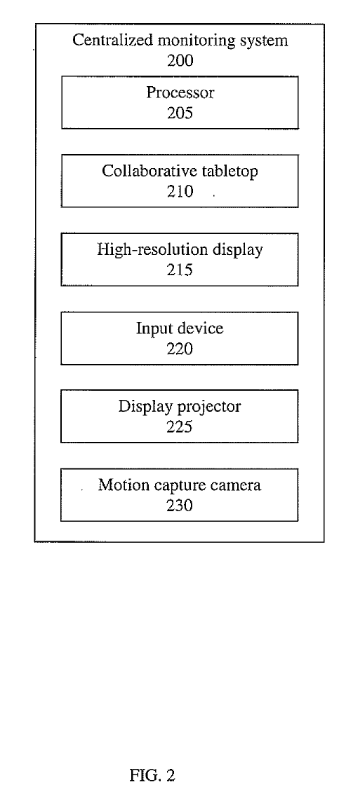

Referring to FIG. 2, the centralized monitoring system 200 is shown in accordance with another embodiment. The centralized monitoring system 200 includes a processor 205, a high-resolution display 215 and an input device 220 that are adapted to enable a single technician to generally simultaneously monitor a plurality of patients.

The high-resolution display 215 is configured for viewing patient data associated with multiple patients being monitored. While depicted as a single device, it should be appreciated that the high-resolution display 215 may comprise multiple displays. In one embodiment, the high-resolution display 215 comprises one or more displays configured to visually convey the patient data for the monitored patients. Alternatively, the high-resolution display 215 may comprise a single display partitioned to visually convey patient data. Further at least one of the high-resolution displays is capable of being calibrated.

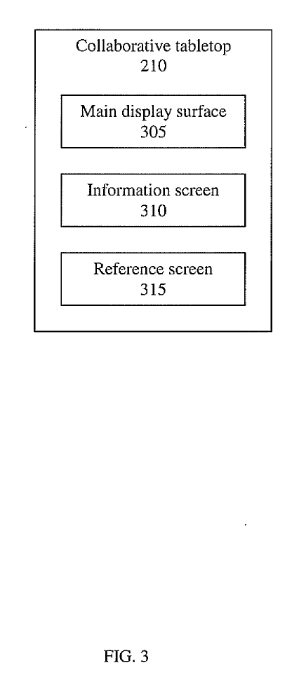

The centralized monitoring system 200 further comprises a collaborative tabletop 210 shows at FIG. 2. The collaborative tabletop is touch sensitive with a predefined resolution. The collaborative tabletop 210 comprises a main display surface 305 for presenting patient data. The main display surface 305 is configured to be used by a plurality of users. In one embodiment, users leverage touch, motion capture, projected information and a combination of low resolution and high resolution, calibrated screens to manipulate information on a patient. User refers to a technician, a supervisor, a clinician, a nurse or a healthcare professional interested in viewing the patient data. This is further explained in conjunction with FIG. 3.

FIG. 3 shows a schematic diagram of the centralized monitoring system 200 In practice, a set of patient waveforms/vitals can be configured to exist in a predetermined location of the main display surface 305. Each user may take control or review details of the patient by touch or by dragging the patient data to another location on the main display surface 305. In addition, each user may be provided with a personalized information screen 310, which may be accessed via touch, and the patient data displayed at one information screen 310 can be dragged or flicked to another information screen on the main display surface 305. Thus, the patient data associated with each patient can be moved to the main display surface 305 or to the information screen 310 designated for the user. Thus, the collaborative tabletop 210 allows multiple users to review, manipulate, and pass key information on a set of monitored patients thereby allowing multiple users to seamlessly manage a set of patients.

In one embodiment, the centralized monitoring system 200 further comprises a display projector 225 configured to display the patient data on a selected portion of the collaborative tabletop 210 at a predetermined resolution. The collaborative tabletop 210 comprises a plurality of high-resolution LCD displays embedded within the main display surface 305, optimized to reflect a projected display from the display projector 225. Skilled artisans shall however appreciate that other display mechanisms may be employed for varied purposes.

A display projected onto the collaborative tabletop 210 by the display projector 225 is analogous to projection by an overhead LCD projector. Alternatively, the display projector 225 is a rear projector (under table) that may as well be oriented above the main display surface 305. The display projector 225 provides the ability to display any part of the patient data (including waveforms) at a desired location on the main display surface 305 at a predetermined resolution. However, the predetermined resolution is limited by the resolution of the display projector 225. In one exemplary embodiment, operational icons or one or more folders comprising patient data may not be actively manipulated as it is limited by the resolution of the display projector 225.

The centralized monitoring system 200 further comprises a motion capture camera 230. The motion capture camera 230 is configured to track the movement of each of the plurality of users, as the user moves the patient data around the collaborative tabletop 210 (or between the information screens 310). Further, the motion capture camera 230 can be configured to work in conjunction with one or touch sensors. In an alternative embodiment, the entire collaborative tabletop 210 may be designed to be touch sensitive with a predetermined resolution. This alleviates the need for the motion capture camera 230.

In one embodiment, blob analysis, infrared detection, capacitance or pressure may be used to identify touch sensation on the collaborative tabletop 210. Skilled artisans shall however appreciate that the collaborative tabletop 210 may leverage other embodiments of software design that does not rely on multi-touch.

The input device 220 is coupled to the processor 205. In a non-limiting manner, the input device 220 may include a keyboard, a mouse, a joystick, a touch pad, etc. In one embodiment, the collaborative tabletop 210 may include areas with dedicated keyboards (either mechanical or touch sensitive) optimized for text input. This eliminates the dependency on the display projector 225 to display a virtual keyboard. In another embodiment, the keyboard may as well be displayed on a touch sensitive surface thereby allowing specific context key mapping and images related to current context. For example, displaying a “silence alarm” button on the keyboard.

The information screen 310 is selected to be a high-resolution, calibrated screen that allows the user to review details related to the patient such as waveforms for multiple leads, trends, alarm histories and tasks. In one embodiment, the user may touch a patient data to move the patient data into the information screen 310 designated to the user. In another embodiment, the user may drag the patient data to the work area (information screen 310 designated to the user). In this case, the information screen 310 and display from the display projector 225 may be synchronized by the processor 205 to make the move from low resolution area to high resolution area seamless. Further, specific calibration of the high-resolution screen can easily be accomplished to allow for caliper measurements or other patient review uses that rely on measurements.

Further, in one embodiment, the collaborative tabletop 210 can further comprise a reference screen 315. The reference screen 315 may be configured to function as a folder adapted to store patient data concerning one or more patients The patient data related to a patient can be placed in the reference screen 315 for easy reference and can be moved back to a selected information screen 310 when desired. The provision of the reference screen 315 allows for streamlining workflow. Thus, the centralized monitoring system 200 comprises a combination of high-resolution displays and low-resolution displays to offer improvements in workflow and ease of use without compromising on resolution.

As described above, the centralized monitoring system 200 is controlled by the processor 205 that coordinates and processes the inputs and outputs for multiple screens and input devices 220. In one embodiment, a single processor 205 is configured to monitor input/output and monitoring functions.

In one embodiment, the processor 205 is programmed with control software that allows for a plurality of resolutions to co-exist (including calibrate displays). The control software is adapted to translate display elements into appropriate coordinates on the main display surface 305 while scaling resolution for individual workspace (information screen 310) and maintain high resolution for high-resolution displays 215. Further, the control software is adapted to snap to a location for selected function elements. For example, a waveform display may be constrained to X,Y coordinates in a work space that corresponds to high-resolution displays 215.

In another embodiment, the centralized monitoring system 200 can comprise a plurality of processors. One of the processors may be configured exclusively for input/output function, while leveraging another processor for core monitoring functions. In either case, the processor 205 provides an abstraction of I/O functions and manages resolution changes and seamless merging of display information. The processor 205 ensures that screens requiring high resolution are placed in appropriate work areas.

In yet another embodiment, a method of displaying patient data in the collaborative tabletop 210 is provided. The method comprises steps of implementing the main display surface 305 for presenting the patient data and implementing at least one information screen 310 configured to be used by a single user. The method may be embodied in the form of a computer implemented process or a computer program product having computer program code containing instructions embodied in tangible media, such as floppy diskettes, CD-ROMs, hard drives, USB (universal serial bus) drives, non-volatile memory, or any other computer readable storage medium, wherein, when the computer program code is loaded into and executed by a computer, the computer becomes an apparatus for practicing the invention. The method may also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing the invention.

When implemented on a general-purpose microprocessor, the computer program code segments configure the microprocessor to control the display of the patient data. A technical effect of the executable instructions is to facilitate the display of the patient data such that a user may easily manipulate the patient data by dragging the patient data to a desired information screen 310, move the patient data into the reference screen 315 for later review and drag the patient data to a desired workspace on the main display surface 305 of the collaborative tabletop 210.

The centralized monitoring system 200 described herein provides a desktop oriented patient management paradigm that simplifies learning and ease of use. In an illustrative embodiment, with appropriate software, a user may easily manipulate patient data by dragging the patient data to a desired information screen 310, move the patient data into the reference screen 315 for later review and drag the patient data to a desired workspace on the main display surface 305 of the collaborative tabletop 210. In one exemplary embodiment, the patient data can be dropped on a paging bucket or dropped on a waveform report page to capture vitals for a shift report.

Thus, the centralized monitoring system 200 described herein provides a highly interactive environment thereby eliminating the large banks of monitors and fatigue associated with manipulating multiple monitors. The centralized monitoring system 200 comprises a combination of displays that allow multiple users to share work while maintaining a personal workspace. Thus, the centralized monitoring system 200 allows a single individual or multiple individuals to work or collaborate on monitoring patients.

The collaborative tabletop 210 provided herein optimizes workflow for reviewing, selecting and interacting with the patient data by allowing touch interaction in a non-fatiguing way. Each user, as described above, can select patient data using a single touch, review key information on a designated information screen 310 and if desired can file the patient data in the reference screen 315 for future reference.

Further, the collaborative tabletop 210 provided herein can eliminate traditional central displays from nursing stations and create a nursing station that comprises central function as part of the work surface.

In various embodiments, a collaborative tabletop for a centralized monitoring system and a centralized monitoring system employing the collaborative tabletop are described. However, the embodiments are not limited and may be implemented in connection with different applications. The application of the invention can be extended to other areas, For example, monitoring systems. The invention provides a broad concept of displaying data, which can be adapted to display items other than patient data in a variety of applications. Accordingly, the invention is not limited to a patient monitoring environment. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A centralized monitoring system for monitoring at least one patient, the centralized monitoring system comprising:
a processor configured to receive patient data from a plurality of patient monitoring systems;
a collaborative tabletop coupled to the processor, the collaborative tabletop configured to visually convey the patient data;
at least one high-resolution display configured to display patient data associated with a plurality of patients; and
an input device coupled to the processor, the input device configured for inputting at least one of textual data, graphical data and image data associated with the patient.

2. The centralized monitoring system of claim 1, wherein the collaborative tabletop comprises:
a main display surface for presenting the patient data, the main display surface configured to be used by a plurality of users; and
at least one information screen configured to be used by a single user.

3. The centralized monitoring system of claim 2, wherein the collaborative tabletop is configured for sensing at least one of a touch, drag, motion capture, a combination of low resolution and high resolution calibrated screens, and projected information to manipulate the patient data.

4. The centralized monitoring system of claim 2, further comprises a display projector configured to display the patient data on a selected portion of the collaborative tabletop at a predetermined resolution.

5. The centralized monitoring system of claim 4, wherein the predetermined resolution does not exceed the resolution of the display projector.

6. The centralized monitoring system of claim 4, wherein the collaborative tabletop is coupled to the display projector and is further configured to reflect a projected display from the display projector.

7. The centralized monitoring system of claim 4, wherein the display projector is a rear projector.

8. The centralized monitoring system of claim 4, wherein the information screen is synchronized with the display projector thereby aiding the movement of the patient data from a low resolution area to a high resolution area.

9. The centralized monitoring system of claim 1, where at least one of the high-resolution displays is capable of being calibrated.

10. The centralized monitoring system of claim 2, wherein the collaborative tabletop further comprises a plurality of touch sensors.

11. The centralized monitoring system of claim 10, further comprises a motion capture camera configured to work in conjunction with the touch sensors to track movement of a user.

12. The centralized monitoring system of claim 1, wherein the input device is one of mechanical and touch sensitive.

13. The centralized monitoring system of claim 1, wherein the processor is further configured to coordinate and process the inputs and outputs for one or more high-resolution displays and the input device.

14. A collaborative tabletop for a centralized monitoring system, the collaborative tabletop comprising:
a main display surface for presenting patient data, the main display surface configured to be used by a plurality of users; and
at least one information screen configured to be used by a single user.

15. The collaborative tabletop of claim 14, is configured for sensing at least one of a touch, drag, motion capture, a combination of low resolution and high resolution calibrated screens and projected information to manipulate the patient data.

16. The collaborative tabletop of claim 14, further comprises a plurality of touch sensors configured to track onscreen movement of each of the plurality of users.

17. The collaborative tabletop of claim 16, is touch sensitive with a predefined resolution.

18. The collaborative tabletop of claim 16, wherein at least one of blob analysis, infrared detection, capacitance, and pressure is used to track motion on the collaborative tabletop.

19. The collaborative tabletop of claim 14, wherein the information screen is configured to be a high-resolution, calibrated display enabling the user to view and analyze the patient data.

20. The collaborative tabletop of claim 14, further comprises a reference screen, the reference screen being configured to function as a folder adapted to store the patient data associated with one or more patients.

21. A method of displaying patient data in a collaborative tabletop, the method comprising:
implementing a main display surface for presenting patient data, the main display surface configured to be used by at least two users; and
implementing at least one information screen configured to be used by a single user.

22. The method of claim 21, further comprising employing a plurality of touch sensors to track onscreen movement of each of the plurality of users.
